(19) United States
(12) Patent Application Publication
(10) Pub. No.: US 2011/0184072 A1
(43) Pub. Date: Jul. 28, 2011

(54) FORMULATION INTENDED TO IMPROVING THE BIOAVAILABILITY OF A HYDROPHOBIC MOLECULE

(76) Inventor: Laurent Pechere, Marseille (FR)

(21) Appl. No.: 13/054,776

(22) PCT Filed: Jul. 16, 2009
(86) PCT No.: PCT/FR2009/000868

§ 371 Date: Apr. 14, 2011

(30) Foreign Application Priority Data
Jul. 18, 2008 (FR) 08 04098

(51) Int. Cl. A61K 31/05; A61P 39/06

(52) U.S. Cl. 514/734

(57) ABSTRACT

The invention relates to a formulation intended to improve the bioavailability by intestinal absorption of polyphenols, comprising at least one polyethylene glycol (and/or functional equivalent(s)) and at least one glycol ether (and/or functional equivalent(s)). The invention is particularly well suited to improving the bioavailability of hydroxystilbenes, particularly 3,5,4′-trihydroxystilbene or resveratrol.

The present invention relates to the field of formulations intended to improve the intestinal absorption of a hydrophobic molecule otherwise called lipophilic.

By intestinal absorption of a hydrophobic molecule, is meant according to the present text the ability of said lipophilic molecule to pass through the gastro-intestinal barrier without having to be metabolized beforehand and to be available in the circulation.

A molecule is called hydrophobic or lipophilic when it is soluble in body fat, but insoluble in water. A hydrophobic molecule does not have the ability to create hydrogen bonds with water molecules. It is also often called apolar, or of weak polarity, which signifies that it cannot create electrostatic interactions with water. In fact, the solubility of a molecule in a solvent generally depends on the interactions that it can have with the solvent. A hydrophobic molecule is therefore a molecule which cannot physically interact with water. It is then generally often soluble in organic solvents.

At present the active ingredients (Al) in development are most often apolar and belong to classes II (low solubility but permeable) and IV (low solubility and low permeability) of the biopharmaceutical classification [Amidon et al., G. L., 1995, Pharm. Res. 12:413-420.].

These characteristic are unfavourable for obtaining a satisfactory absorption by oral route.

Therefore a requirement remains for a galenic formulation which increases the absorption of hydrophobic molecules. This is one of the purposes of the present invention.

Among the hydrophobic molecules, the polyphenols are compounds that are found in the natural state in the plants of the class of the spermatophytes and particularly in the vine. Such compounds such as for example resveratrol are found in the grape and in wine.

Among the polyphenols, the hydroxystilbenes are found. In the prior art the hydroxystilbenes are used, among other things, as depigmenting agents (JP87-192040), as vasodilator agents (EP 96-830517), as antithrombic agents (JP 05016413), in the treatment of various cardio-vascular diseases (CA 2187990), as agents that inhibit mutagenesis and carcinogenesis (JP 06024967), or also described as antioxidants.

Resveratrol (3,5,4′-trihydroxystilbene) is a polyphenolic phytoalexin. This compound is synthesized by plants and acts as an antifungal in response to infections (Bothrytis cinerea). Resveratrol is found in various plants such as conifers, peanuts, the skin of red grapes, certain leguminous plants and Polygonum cuspidatum.

Resveratrol has therapeutic properties which have been known for a long time in traditional Chinese and Japanese medicine. Resveratrol has been indicated as responsible for the reduction in the cardiovascular risks called the “French Paradox”. In fact, a correlation has been established between the consumption of red wine containing high levels of resveratrol, and the reduction in coronary diseases. Numerous scientific studies have demonstrated that resveratrol is an antagonist of the dioxin and aryl hydrocarbon receptor (AhR) (Casper, R. F., et al., Mol. Pharmacol. 1999, 56, 784-790).

Resveratrol also has antioxidant, anti-inflammatory, osteoprotective activities and could have a preventive effect in certain cancers.

In vivo models have been used to study the absorption of resveratrol. In the rat, kinetic studies have been carried out at different times after absorption of red wine. The results indicate that an absorption peak is detected 60 minutes after ingestion. After a short period, resveratrol is detected in the liver and the kidneys (micromolar maximum one hour after absorption). Elimination is very rapid with the kidneys as preferred organs of elimination. Other studies show that serous resveratrol appears after 15 minutes and reduces very rapidly after 30 minutes. Similar work has been carried out in mice. Resveratrol appears to be absorbed to the maximum extent by the duodenum and the elimination of resveratrol is very rapid with maximum absorption at 30 minutes.

It appears from these studies that resveratrol is very rapidly absorbed, metabolized during enterohepatic cycling and eliminated.

Numerous ex vivo and in vitro studies have been carried out on the absorption and metabolism of resveratrol in order to understand the localization of resveratrol in the various organs after its administration and the metabolites produced.

The ex vivo models are represented by perfusions of the small intestine of the rat. This type of study indicates that resveratrol is extracted from the small intestine at a level of 46%, 21% being found at the vascular level and only 2% being found the intestinal level. 40% of this resveratrol is free while 11% is glucurono-conjugated and 3% is in sulphated form. The glucuronide form is that found in the circulatory system while the sulphated form is the form secreted in the intestinal luminal part. This same type of study has been carried out in ileum and perfused colon models.

The results indicate that only a minute portion of the resveratrol is not metabolized. Studies on similar liver or human intestine models indicate that resveratrol is also very quickly metabolized. Resveratrol is therefore absorbed and metabolized with the intestinal level. When resveratrol passes into the circulation, it is largely bound to the serum proteins.

The in vitro studies using cell lines (intestinal line CaCo-2) have confirmed the results obtained on perfused intestinal models.

Finally, since 2003, metabolism and bioavailability studies have been carried out on humans. The principals given indicate that resveratrol reaches a maximum serous concentration after 30 minutes. Beyond 30 minutes, it is rare to find unmetabolized resveratrol. In the best cases, 2% of the resveratrol is found in the plasma in the unchanged form!

All these data suggest that the activity of resveratrol as a therapeutic agent is closely linked to its bioavailability which results from its intestinal absorption and the rapidity of its metabolism.

One of the ways of increasing the concentration of serous unchanged resveratrol is to develop a galenic form which makes it possible to increase its absorption and therefore its bioavailability. This is one of the purposes of the present invention.

Thus a subject of the invention is the use of a formulation intended to improve the absorption, advantageously the intestinal absorption, of polyphenols, comprising at least one polyethylene glycol or one of its functional equivalents and at least one glycol ether or one of its functional equivalents.

A formulation comprising resveratrol, a polyethylene glycol and a glycol ether has been disclosed in the document WO01/30336, but for treating cutaneous problems, by a topical use. As for the document WO2004/071490 it discloses a formulation comprising polyethylene glycol, a glycol ether and KOH, with the aim of increasing the bioavailability of acidic pharmaceutical active ingredients of low solubility. Now it is described in the literature that a such a formulation cannot be used to increase the availability of polyphenols, as it destroy the polyphenols (Nardini M., Cirillo E., Natella F., Mencarelli D., Comisso A, Scaccini S., Food Chemistry, vol. 79, issue 1, October 2004, 16, p. 119-124).

By “polyphenols” is meant according to the invention natural and synthetic polyphenols.

By “synthetic polyphenol” is meant more specifically any polyphenol obtained by chemical synthesis and not by extraction of biological material (plants) as well as any derivative of a natural polyphenol modified by the substitution or addition of atoms to the natural structure. Advantageously, these substitutions are by halogens (Cl—, CF3-) or radicals of general structure R—O— where R is an aliphatic chain or an aromatic ring or a nitrated radical.

By “functional equivalents” is meant according to the invention a compound which, mixed with a polyphenol compound, has the same effects on the latter as a polyethylene glycol and/or a glycol ether.

Thus according to the invention, a formulation can be used comprising a polysorbate as a functional equivalent of polyethylene glycol.

Similarly according to the invention, a formulation can be used comprising glycerine or polyglyceryl-3 dioleate (polyglyceral ester of fatty acids or one of its equivalents) as a functional equivalent of glycol ether.

According to the invention, by polyethylene glycol is meant any polymer corresponding to the formula H(OCH₂CH₂)ₙOH where n is greater than three. in this respect the polyethylene glycols of average molecular weight comprised between approximately 100 and 20,000, preferentially of average molecular weight comprised between approximately 400 and approximately 10,000, very preferentially of average molecular weight comprised between approximately 400 and approximately 600 can be mentioned by way of example.

According to the invention, a polyethylene glycol of a given molecular weight can be used alone or also in a mixture in any proportion with one or more other polyethylene glycols of varied molecular weight or other functional equivalents.

The polyethylene glycols used in the context of the invention, can be presented at ambient temperature either in liquid form, or in semi-solid form depending on their molecular weight. Consequently, these polymers are selected appropriately depending on whether the formulation intended to improve the absorption of the polyphenols according to the invention must be in liquid form or conversely semi-solid form.

According to the invention, the glycol ether can be chosen from diethylene glycol ethers such as for example diethylene glycol alkyl ethers, particularly the (C1-C4) diethylene glycol alkyl ethers, chosen from diethylene glycol methyl ether, diethylene glycol ethyl ether, diethylene glycol propylyl ethers or diethylene is glycol butyl ethers, very particularly diethylene glycol mono-(C1-C4) alkyl ethers chosen from diethylene glycol monomethyl ether, diethylene glycol monoethyl ether, diethylene glycol monopropylyl ethers or diethylene glycol monobutyl ethers.

Among the diethylene glycol alkyl ethers, the methyl and ethyl ethers, in particular the diethylene glycol monoethyl ether are preferred.

The glycol ethers can according to the invention be used alone or combined in any proportions with one (or more) other glycol ether(s) and/or with one (or more) other functional equivalent(s).

According to the invention, the formulation intended to improve the absorption of polyphenols can comprise polyethylene glycol, or one of its functional equivalents such as for example a polysorbate, in a proportion comprised between 20 and 97%, preferentially between 40 and 97% by weight of the total weight of the formulation.

Also according to the invention the formulation intended to improve the absorption of polyphenols can comprise glycol ether, or one of its functional equivalents such as for example glycerine or polyglyceryl-3 dioleate (polyglyceral ester of fatty acids or one of its equivalents), in a proportion comprised between 2 and 79%, preferentially between 2 and 59% by weight of the total weight of the formulation.

A particularly preferred formulation according to the invention comprises between 50 and 93% polyethylene glycol, or one of its functional equivalents such as for example a polysorbate, between 3 and 46% glycol ether, or one of its functional equivalents such as for example glycerine or polyglyceryl-3 dioleate (polyglyceral ester of fatty acids or one of its equivalents), and a sufficient quantity of water to make up 100%.

According to a variant of the invention, the formulation can also comprise at least one emulsifier. Advantageously according to the invention the emulsifier can be a polysorbate, even more advantageously a polysorbate chosen from Polysorbate 20 (Tween 20 or sorbitan polyoxyethylene (20) monolaurate), Polysorbate 40 (Tween 40 or sorbitan polyoxyethylene (20) monopalmitate), Polysorbate 60 (Tween 60 or sorbitan polyoxyethylene (20) monostearate), or Polysorbate 80 (Tween 80 or sorbitan polyoxyethylene (20) monooleate).

The formulation according to the invention is particularly suitable to be used for improving the absorption, advantageously the intestinal absorption, of polyphenols, particularly hydroxystilbenes such as for example those of Formula (I),

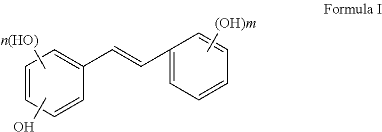

in which n is an integer comprised between 0 and 4 inclusive and m is an integer comprised between 0 and 5 inclusive. These compounds can be in Cis or Trans form. According to the invention, the term hydroxystilbene covers the compounds of Formula I as well as their hydroxyalkylated derivatives.

Among the hydroxystilbenes, there can be mentioned the mono, di, tri, tetra, penta, hexa, hepta, octo, nonahydroxystilbenes, or also their hydroxyalkylated derivatives, for example 4′-hydroxystilbene, 2′,4′-dihydroxystilbene, 3′,4′-dihydroxystilbene, 4,4′-dihydroxystilbene, 2′,4′,4-trihydroxystilbene, 3′,4′,4-trihydroxystilbene, 2,4,4′-trihydroxystilbene, 3,4,4′-trihydroxystilbene, 3,5,4′,-trihydroxystilbene, 2′,3,4-trihydroxystilbene, 2,3′,4-trihydroxystilbene, 2′,2,4′-trihydroxystilbene, 2,4,4′,5-tetrahydroxystilbene, 2′,3.4′,5-tetrahydroxystilbene, 2,2′,4,4′-tetrahydroxystilbene, 3,3′,4′,5-tetrahydroxystilbene, 2,3′,4,4′-tetrahydroxystilbene, 3,3′,4,4′-tetrahydroxystilbene, 3,3′,4′,5,5′-pentahydroxystilbene, 2,2′,4,4′,6-pentahydroxystilbene, 2,3′,4,4′,6-pentahydroxystilbene, 2,2′,4,4′,6,6′-hexahydroxystilbene.

Preferentially, 3,5,4′-trihydroxystilbene or resveratrol is used according to the invention.

In a preferred composition according to the invention, intended for oral route, the use of a support that can be ingested is favoured. The composition according to the invention can take the form of sugar-coated tablets, gelatin capsules, gels, emulsion, tablets, capsules or other galenic forms which can be used per os. These forms are produced by the usual processes known to a person skilled in the art.

According to a particular embodiment of the invention, the composition can be formulated in encapsulated form so as to significantly improve the shelf life of the active ingredient.

Other characteristics and advantages of the invention will become more apparent from the following examples, given by way of a non-limitative illustration. In what follows, or in the above, the proportions are given in percentage by weight, unless otherwise indicated.

### EXAMPLE 1

### Preparation of a Composition Incorporating the Formulation According to the Invention

The following composition is prepared which is presented in liquid form:

| | | |
|---|---|---|
| Transcutol P ® | 25 | mg |
| PEG 600 | 455 | mg |
| Resveratrol | 20 | mg |

Resveratrol is dissolved in the presence of Transcutol P and PEG 600 which have been weighed beforehand. The mixture is stirred until a translucent and clear liquid phase is obtained. Then it is encapsulated in gelatin capsules which are standard for pharmaceutical or food use.

### EXAMPLE 2

### Measurement of the Blood Level of Resveratrol after Ingestion

A capsule as prepared in Example 1 is ingested by a volunteer from whom a blood sample has been taken beforehand. A second blood sample from the volunteer is taken 4 hours after ingestion.

The blood samples are then analyzed by liquid chromatography-mass spectrometry according to the usual protocols.

In this way the level of resveratrol in the blood before and after ingestion is determined.

The trial is carried out on 3 different volunteers.

| | Concentration of resveratrol in the | | Level of |
|---|---|---|---|
| | blood in μg/L | | increase in the |
| Individuals | T = 0 h | T = 4 h | concentration |
| 1 | 79 | 285 | 3.6 |
| 2 | 23 | 396 | 17.2 |
| 3 | 29 | 295 | 10.4 |

These trials show that resveratrol is still present at a clearly higher concentration 4 hours after ingestion of a composition according to the invention.

### EXAMPLE 3

### Measurement of the Level of Resveratrol in the Blood after Ingestion

Two capsules as prepared in Example 1 are ingested by 5 volunteers from whom a blood sample has been taken beforehand. A second blood sample is taken from the volunteers 30 minutes after ingestion. A third blood sample is taken 5 hours after ingestion. Another individual ingests 40 mg of resveratrol in standard form (gelatin capsule).

The blood samples are then analyzed by liquid chromatography-mass spectrometry according to the usual protocols.
In this way the level of resveratrol in the blood before and after ingestion is determined.
The table below indicates the average concentration values.

| | Average concentration of | | |
|---|---|---|---|
| | resveratrol in the blood (nM) | | |
| | T = 0 h | T = 30 min | T = 5 h |
| Standard gelatin capsule | 0 | 422 | 0 |
| formula | | | |
| Capsule formula according | 0 | 5295 | 491 |
| to the present invention | | | |
| (PEG + Transcutol) | | | |

These trials show that resveratrol is still present in a clearly high concentration 5 hours after ingestion of a composition according to the invention.

1. A method of improving the absorption of at least one polyphenol by a subject, comprising administering an effective amount of a formulation comprising at least one polyethylene glycol or a functional equivalent and at least one glycol ether or a functional equivalent.

2. The method according to claim 1, characterized in that the polyethylene glycol is a polymer corresponding to the formula H(OCH₂CH₂)ₙ OH where n is greater than three.

3. The method according to claim 1, characterized in that the polyethylene glycol is chosen from polyethylene glycols of average molecular weight between approximately 100 and 20,000.

4. The method according to claim 1, characterized in that the polyethylene glycol or one of its functional equivalents, is in a proportion comprised between 20 and 97%, by weight of the total weight of said formulation.

5. The method according to claim 1, characterized in that the glycol ether is a diethylene glycol ether.

6. The method according to claim 1, characterized in that the glycol ether or one of its functional equivalents, is in a proportion comprised between 2 and 79% by weight of the total weight of said formulation.

7. The method according to claim 1, characterized in that the formulation comprises between 50 and 93% polyethylene glycol or one of its functional equivalents, 3 and 46% glycol ether or one of its functional equivalents and a sufficient quantity of water to make up 100%.

8. The method according to claim 1, characterized in that the formulation comprises a functional equivalent of polyethylene glycol and a functional equivalent of glycol ether.

9. The method according to claim 1, characterized in that in the formulation the functional equivalent of polyethylene glycol is a polysorbate.

10. The method according to claim 1, characterized in that in the formulation the functional equivalent of glycol ether is glycerine or polyglyceryl-3 dioleate.

11. The method of claim 1, which further improves the absorption, a of at least one hydroxystilbene.

12. The method according to claim 11, characterized in that the hydroxystilbene corresponds to Formula 1 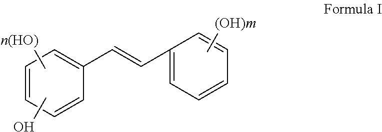
in which n is an integer comprised between 0 and 4 inclusive and m is an integer comprised between 0 and 5 inclusive, in Cis or Trans form, or a hydroxyalkylated derivative thereof.

13. The method according to claim 11, characterized in that at least one hydroxystilbene is a mono, di, tri, tetra, penta, hexa, hepta, octo, or nonahydroxystilbene, or a hydroxyalkylated derivative thereof.

14. The method of claim 1 wherein such absorption is intestinal absorption.

15. The method of claim 1 wherein the polyethylene glycol is chosen from polyethylene glycols of average molecular weight between approximately 400 and approximately 10,000.

16. The method of claim 1 wherein the polyethylene glycol is chosen from polyethylene glycols of average molecular weight between approximately 400 and approximately 600.

17. The method of claim 1 in which the glycol ether is a C1-C4 diethylene glycol alkyl ether.

18. The method of claim 1 in which the glycol ether is a diethylene glycol mono (C1-C4) alkyl ether.

19. The method of claim 1 in which the glycol ether is a diethylene glycol monoethyl ether.

20. The method of claim 11 wherein at least one hydroxystilbene is selected from the group consisting of 4′-hydroxystilbene, 2′,4′-dihydroxystilbene, 3′,4′-dihydroxystilbene, 4.4′-dihydroxystilbene, 2′,4′,4-trihydroxystilbene, 3′,4′,4-trihydroxystilbene, 2,4,4′-trihydroxystilbene, 3,4,4′-trihydroxystilbene, 3,4′,5-trihydroxystilbene, 2′,3,4-trihydroxystilbene, 2,3′,4-trihydroxystilbene, 2′,2,4′-trihydroxystilbene, 2,4,4′,5-tetrahydroxystilbene, 2′,3,4′,5-tetrahydroxystilbene, 2,2′,4,4′-tetrahydroxystilbene, 3,3′,4′,5-tetrahydroxystilbene, 2,3′,4,4′-tetrahydroxystilbene, 3,3′,4,4′-tetrahydroxystilbene, 3,3′,4′,5,5′-pentahydroxystilbene, 2,2′,4,4,6-pentahydroxystilbene, 2,3′,4,4′,6-pentahydroxystilbene, 2,2′,4,4′,6,6′-hexahydroxystilbene.

21. The method of claim 11 wherein at least one hydroxystilbene is 3,4′,5-trihydroxystilbene (resveratrol).

22. The method of claim 11 wherein the absorption of the at least one hydroxystilbene is intestinal.

23. The method of claim 6 in which the glycol ether or one of its functional equivalents is in a proportion comprised between 2 and 59%; by weight of the total weight of said formulation.

24. The method of claim 4 in which the polyethylene glycol or one of its functional equivalents is in a proportion comprised between 40 and 97% by weight of the total weight of said formulation.

25. The method of claim 9 in which the polysorbate is selected from the group consisting of Polysorbate 20, Polysorbate 40, Polysorbate 60 and Polysorbate 80.
